(19) United States
(12) Patent Application Publication
(10) Pub. No.: US 2021/0331150 A1
(43) Pub. Date: Oct. 28, 2021

(54) SARS-COV-2 SUSCEPTIBILITY DETECTION

(71) Applicant: Kevin D. FIALKO, La Jolla, CA (US)

(72) Inventor: Kevin D. FIALKO, La Jolla, CA (US)

(21) Appl. No.: 17/238,795

(22) Filed: Apr. 23, 2021

Related U.S. Application Data
(60) Provisional application No. 63/015,089, filed on Apr. 24, 2020.

(51) Int. Cl. B01L 1/00; C07C 205/11

(52) U.S. Cl. CPC B01L 1/00; C07C 205/11

(57) ABSTRACT

A Covid-19 susceptibility testing device including a receiving chamber configured to receive a human bodily fluid and a reaction medium configured to indicate Covid-19 susceptibility when concentration of an analyte exceeds a predetermined threshold in the human bodily fluid.

### CROSS-REFERENCE TO RELATED APPLICATION

The present application claims the right of priority to U.S. Provisional Patent Application No. 63/015,089 having a filing date of Apr. 24, 2020, the contents of which are hereby incorporated by reference in their entirety.

### FIELD OF THE INVENTION

The present invention relates to detection of risk level regarding an individual's susceptibility to infection by Covid-19.

### BACKGROUND

Devices, such as early detection pregnancy tests (EPT), may allow for home detection of pregnancy based on hormone concentration in a female individual's urine. However, specific application of coronavirus (e.g., Covid-19) has not been previously addressed in the known prior art.

### BRIEF DESCRIPTION OF THE DRAWINGS

Various aspects of the present invention are illustrated by way of example, and not by way of limitation, in the accompanying drawings, wherein:

FIG. 1 depicts a diagram of a SARS-CoV-2 virus cell beside a human cell in accordance with the principles of the present invention;

FIG. 2 depicts a diagram of a home test device for detection of elevated levels of ACE2 in accordance with the principles of the present invention;

FIG. 3a depicts a diagram of ACE2 containing human cells in suspension above a substrate-bound test antibody comprising an S-protein in accordance with the principles of the present invention;

FIG. 3b depicts a diagram of the ACE2-containing human cells bound to the S-protein of the substrate-bound test antibody with the addition of enzyme-containing free test antibody bound to S-proteins in accordance with the principles of the present invention;

FIG. 3c depicts a diagram of ACE2 containing human cells bound to the substrate and further bound to the S-proteins of enzyme-containing free test antibodies in accordance with the principles of the present invention;

FIG. 4 depicts a block-level diagram of a method of testing for elevated ACEII in an individual as illustrated in FIGS. 3a-3c in accordance with the principles of the present invention;

FIG. 5a depicts a diagram of an alternate embodiment of a reaction mechanism for testing for elevated ACE2 of an individual in accordance with the principles of the present invention;

FIG. 5b depicts a diagram of the alternate embodiment reaction mechanism of FIG. 5a having a positive result in accordance with the principles of the present invention; and

FIG. 6 depicts a block-level diagram of the method of the alternate embodiment of testing depicted in FIGS. 5a and 5b in accordance with the principles of the present invention.

### DETAILED DESCRIPTION

### Description

The structures and methods described herein may include a device that may be used at home by an individual to test for concentration and/or elevated levels of ACE2 receptors relative to a general population, as elevated ACE2 receptors may be found to correspond to increased susceptibility of the individual to human coronavirus, such as SARS-CoV-2 (e.g. the virus that causes COVID-19). The individual may consult a doctor for prophylaxis, such as prescription of hydroxychloroquine, azithromycin, and/or zinc. In some embodiments, the present invention may include assessment of the individual's age and/or underlying health risk factors, such as diabetes, high blood pressure, and/or heart health history.

In some embodiments, a device comprising a reaction chamber may receive bodily fluid from the individual. The elevated presence of ACE2 receptors in the bodily fluid may be indicated by color change of a chromogenic substrate.

Embodiments of the present invention may comprise an at-home instant result kit, with a color code indicator and tongue swab. By way of example, a color change to red or other operable color, after the tongue swab, may indicate elevated levels of ACE2 receptors of the individual. The elevated levels of ACE2 may be indicative of increase susceptibility to SARS-CoV-2 infection. The individual may then call and inform the individual's physician regarding elevated ACE2 receptors.

The detailed description set forth below in connection with the appended drawings is intended as a description of various embodiments of the invention and is not intended to represent the only embodiments in which the invention may be practiced. The detailed description includes specific details for the purpose of providing a thorough understanding of the invention. However, it will be apparent to those skilled in the art that the invention may be practiced without these specific details. In some instances, well known structures and components are shown in block diagram form in order to avoid obscuring the concepts of the invention.

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a controller, a general purpose computer, a special purpose computer, or other programmable data processing apparatus to produce a machine, such that the instructions, which executed via the processor of the controller or other programmable data processing apparatus, create means for implementing the functions/acts specified in the flowchart and/or block diagram block or blocks.

The following description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean “one and only one” unless specifically so stated, but rather “one or more.” Similarly, references to an element in the singular in the description mean “one or more” unless specifically stated otherwise. All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As will be appreciated by one skilled in the art, aspects of the present invention may be embodied as a method, system, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product on a computer-usable storage medium having computer-usable program code embodied in the medium.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. Aspects of the invention were chosen and described in order to best explain the principles of the invention and the practical application, and to enable others of ordinary skill in the art to understand the invention for various embodiments with various modifications as are suited to the particular use contemplated.

With respect to the present application, “congener” means a chemical constituent of the same kind or category, having similar chemical properties, binding affinity, and functionality, and minimal structural changes, relative to the referenced chemical compound.

With respect to the present application, “about” or “approximately” means within plus or minus one at the last reported digit. For example, about 1.00 means 1.00±0.01 unit.

With respect to the present application, “around” used in conjunction with a numeral measurement means within plus or minus one unit. For example, around 50% means 49%-51%. For example, around 11.01 units means 10.01-12.01.

With respect to the present description “and” and “or” shall be construed as conjunctively or disjunctively, whichever provides the broadest disclosure in each instance of the use of “and” or “or.”

Embodiments of the present invention may include ELISA tests capable of determining ACE2 concentration in different bodily fluids. Examples may include testing for elevated ACE2 concentration above the average population ACE2 levels in various human bodily fluids to determine Covid-19 susceptibility. However, such tests may have historically relied on the use of analytical tools such as spectroscopy to determine the actual concentration. Thus, embodiments of the present invention may provide for a test that may be read by the naked eye. For example, a colorimetric test for simplified lab use or at home use.

The reaction indication, such as a binary response, may be triggered if the ACE2 concentration is above the average human concentration in the particular bodily fluid. The response may not necessarily be triggered if the ACE2 concentration is at or below the average concentration in the particular bodily fluid. For urine, the mean urine concentration of ACE2 may be 75.24 ng/ml. Furthermore, the average ACE2 concentrations may further be used in tissue as follows: lung extract (26 ng/ml), heart extract (31 ng/ml), fetal intestine extract (115 ng/ml), kidney extract (94 ng/ml), and testes extract (146 ng/ml). Similarly, human bronchial epithelial cells ACE2 concentration may be slightly greater than 100 ng/ml.

A filter may be used in some embodiments to remove excess debris or analyte that does not necessarily correspond to elevated ACE2 and/or Covid-19 susceptibility. This filter may screen out bacteria that could also be coated with small molecules, which bind and hold ACE2 in place and prevent it from reaching the reaction chamber. The purpose of this coating would be to only allow ACE2 to pass through it if the fluid contains an above average concentration of ACE2.

Using urine as an example, a user may collect their urine in a separate container. Add in a specified amount of urine (e.g., 10 drops) from the separate container to a device receiving chamber, which may comprise an absorbent pad connected to a filter, in some embodiments. Because 10 drops equal 0.50 mL, the filter may be configured with enough small molecules to collect 37.62 ng of ACE2. Once the filter was saturated with this amount of ACE2, any additional ACE2 present would pass through the filter to reach the reaction chamber. At the reaction chamber, the available ACE2 may react with a chromogenic substrate, producing a color which indicates a positive result.

The Chromogenic Substrate and the Binding Site

The binding site of ACE 2 (with a HIS-LEU-like small molecule located inside) may be shown below.

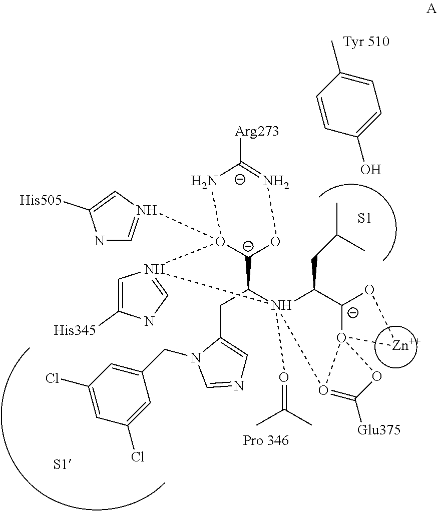

ACE2 has been shown to be a carboxypeptidase, such that it may cleave peptide bonds at the carboxy-terminal of a peptide. Various examples exist of molecules capable of binding to this site. One of the central designs of these molecules includes a carboxylate group needed to coordinate with Zinc. Further, HIS-LEU like molecules have been shown effective at preventing cleavage.

Regarding chromogenic substrates for indication of a positive result of Covid-19 susceptibility due to increased ACE2, there are several options. For example, the reaction may be conducted using a sandwich ELISA (e.g. which may be substantially similar to the ThermoFisher Scientific Kit Catalog #BMS2323 for detection of antibodies). Alternatively, in embodiments using a filter, the filter may remove any individual amino acids found in the human bodily fluid sample, then have ACE2 react with ACE to produce leucine. In turn, ninhydrin may be added to the solution to produce a colored reaction if leucine is present. Further embodiments include using the chromogenic substrate, APK-DNP (Mca-Ala-Pro-Lys(Dnp) [Mca=(7-methoxycoumarin-4-yl)acetyl;Dnp=2,4-dinitrophenyl]; C₃₂H₃₇N₆O₁₂; available from Enzo Life Sciences BML-P163-0001), allow cleavage of DNP by ACE2. Then, proceed with a Brady's reagent (e.g. 2,4-Dinitrophenylhydrazine) reaction with DNP to form a colored precipitate.

Additional embodiments include use of a chromogenic substrate ending with Ala-Pro-Lys-pNA. pNA (paranitroaniline), which may be similar in size to DNP (dinitrophenol), the fluorescent leaving group in the peptide papers on box. If the reaction proceeds as expected, pNA would produce a colored reaction indicating a positive result.

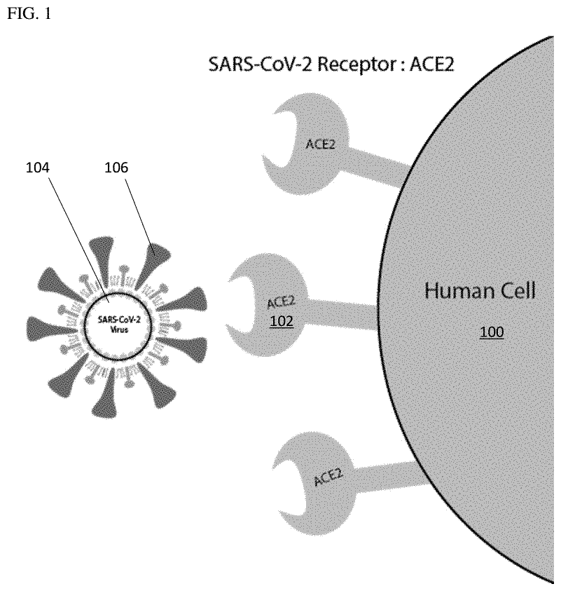

FIG. 1 depicts a diagram of a SARS-CoV-2 virus cell 104 beside a human cell 100 in accordance with embodiments of the present invention. The SARS-CoV-2 cell 104 may comprise S-proteins 106 that adorn the virus cell 104. The human cell 100 may comprise ACE2 integral transport proteins 102. The S-proteins 106 may bind with the ACE2 proteins to cause the human cell to internalize the virus cell 104 by receptor-mediated endocytosis.

Severe acute respiratory syndrome coronavirus 2 (SARS-CoV-2) is the virus strain that causes coronavirus disease 2019 (COVID-19), a respiratory illness. This virus is depicted as virus cell 104. It may be colloquially known as the coronavirus, and was previously referred to by its provisional name 2019 novel coronavirus (2019-nCoV). SARS-CoV-2 may be a positive-sense single-stranded RNA virus. SARS-CoV-2 is contagious in humans, and the World Health Organization (WHO) has designated the ongoing pandemic of COVID-19 a Public Health Emergency of International Concern. Therefore, avoidance of infection is desirable for an individual. Embodiments of the present invention relate to risk factor detection related to COVID-19 and SARS-CoV-2.

Taxonomically, SARS-CoV-2 is a strain of Severe acute respiratory syndrome-related coronavirus (SARSr-CoV). It is believed to have zoonotic origins and has close genetic similarity to bat coronaviruses, suggesting it emerged from a bat-borne virus. An intermediate animal reservoir such as a pangolin is also thought to be involved in its introduction to humans. The virus shows little genetic diversity, indicating that the spillover event introducing SARS-CoV-2 to humans is likely to have occurred in late 2019.

Epidemiological studies estimate each infection results in 1.4 to 3.9 new ones when no members of the community are immune and no preventive measures taken. The virus is primarily spread between people through close contact and via respiratory droplets produced from coughs or sneezes. It mainly enters human cells by binding to the receptor angiotensin converting enzyme 2 (ACE2).

Each SARS-CoV-2 virion, such as virus cell 104, may be approximately 50-200 nanometers in diameter. SARS-CoV-2 may comprise four structural proteins, known as the S (spike) 106, E (envelope), M (membrane), and N (nucleocapsid) proteins; the N protein holds the RNA genome, and the S, E, and M proteins together create the viral envelope. The spike protein, which has been imaged at the atomic level using cryogenic electron microscopy, is the protein responsible for allowing the virus to attach to and fuse with the membrane of a host cell, such as human cell 100.

Protein modeling experiments on the spike protein of the virus may have suggested that SARS-CoV-2 may have sufficient affinity to the receptor angiotensin converting enzyme 2 (ACE2) on human cells to use them as a mechanism of cell entry. By 22 Jan. 2020, a group in China working with the full virus genome and a group in the United States using reverse genetics methods independently and experimentally demonstrated that ACE2 could act as the receptor for SARS-CoV-2. Studies have shown that SARS-CoV-2 may have a higher affinity to human ACE2 than the original SARS virus strain. SARS-CoV-2 may also use basigin to assist in cell entry.

Initial spike protein priming by transmembrane protease, serine 2 (TMPRSS2) may be essential for entry of SARS-CoV-2. After a SARS-CoV-2 virion attaches to a target cell, the cell's protease TMPRSS2 may cut open the spike protein of the virus, exposing a fusion peptide. The virion may then releases RNA into the cell, forcing the cell to produce copies of the virus that may be disseminated to infect more cells. SARS-CoV-2 may produce at least three virulence factors that may promote shedding of new virions from host cells and inhibit immune response.

Angiotensin-converting enzyme 2 (ACE2) 102 is an enzyme attached to the cell membranes of human cells 100 in the lungs, arteries, heart, kidney, and intestines. ACE2 102 lowers blood pressure by catalysing the hydrolysis of angiotensin II (a vasoconstrictor peptide) into angiotensin (1-7) (a vasodilator). ACE2 102 counters the activity of the related angiotensin-converting enzyme (ACE) by reducing the amount of angiotensin-II and increasing angiotensin (1-7) making it a promising drug target for treating cardiovascular diseases.

ACE2 also serves as the entry point into cells for some coronaviruses. The human version of the enzyme is often referred to as hACE2. Angiotensin-converting enzyme 2 is a zinc containing metalloenzyme located on the surface of endothelial and other cells. ACE2 protein contains an N-terminal peptidase M2 domain and a C-terminal collectrin renal amino acid transporter domain.

ACE2 is known to be present in most organs. ACE2 is attached to the cell membrane of mainly lung type II alveolar cells, enterocytes of the small intestine, arterial and venous endothelial cells and arterial smooth muscle cells in most organs. ACE2 mRNA expression is also found in the cerebral cortex, striatum, hypothalamus, and brainstem.

The primary function of ACE2 is to act as a counterbalance to ACE. ACE cleaves angiotensin I hormone into the vasoconstricting angiotensin II. ACE2 in turn cleaves the carboxyl-terminal amino acid phenylalanine from angiotensin II (Asp-Arg-Val-Tyr-Ile-His-Pro-Phe) and hydrolyses it into the vasodilator angiotensin (1-7), (H-Asp-Arg-Val-Tyr-Ile-His-Pro-OH). ACE2 can also cleave a number of other peptides including [des-Arg9]-bradykinin, apelin, neurotensin, dynorphin A, and ghrelin. ACE2 also regulates the membrane trafficking of the neutral amino acid transporter SLC6A19 and has been implicated in Hartnup's disease.

As a transmembrane protein, ACE2 serves as the main entry point into cells for some coronaviruses, including HCoV-NL63; SARS-CoV (the virus that causes SARS); and SARS-CoV-2 (e.g. the virus that causes COVID-19). More specifically, the binding of the spike S1 protein of SARS-CoV and SARS-CoV2 to the enzymatic domain of ACE2 on the surface of cells results in endocytosis and translocation of both the virus and the enzyme into endosomes located within cells. This entry process also requires priming of the S protein by the host serine protease TMPRSS2, the inhibition of which is under current investigation as a potential therapeutic.

This has led some to hypothesize that decreasing the levels of ACE2, in cells, might help in fighting the infection. On the other hand, ACE2 has been shown to have a protective effect against virus-induced lung injury by increasing the production of the vasodilator angiotensin 1-7. Furthermore, according to studies conducted on mice, the interaction of the spike protein of the coronavirus with ACE2 induces a drop in the levels of ACE2 in cells through internalization and degradation of the protein and hence may contribute to lung damage.

Both ACE inhibitors and angiotensin receptor blockers (ARBs) that are used to treat high blood pressure have been shown in rodent studies to upregulate ACE2 expression hence may affect the severity of coronavirus infections. However, multiple professional societies and regulatory bodies have recommended continuing standard ACE inhibitor and ARB therapy. A systematic review and meta-analysis published on Jul. 11, 2012, found that “use of ACE inhibitors was associated with a significant 34% reduction in risk of pneumonia compared with controls.” Moreover, “the risk of pneumonia was also reduced in patients treated with ACE inhibitors who were at higher risk of pneumonia, in particular those with stroke and heart failure. Use of ACE inhibitors was also associated with a reduction in pneumonia related mortality, although the results were less robust than for overall risk of pneumonia.”

ACE2 is a single-pass type I membrane protein, with its enzymatically active domain exposed on the surface of cells in lungs and other tissues. The extracellular domain of ACE2 is cleaved from the transmembrane domain by another enzyme known as sheddase, and the resulting soluble protein is released into the blood stream and ultimately excreted into urine.

ACE2, also called ACEH (ACE homolog), is an integral membrane protein and a zinc metalloprotease of the ACE family that also includes somatic and germinal ACE. Human ACE-2 has about 40% amino acid identity to the N- and C-terminal domains of human somatic ACE. The predicted human ACE-2 protein sequence consists of 805 amino acids, including a N-terminal signal peptide, a single catalytic domain, a C-terminal membrane anchor, and a short cytoplasmic tail. ACE-2 cleaves angiotensins I and II as a carboxypeptidase. ACE-2 mRNA is found at high levels in testis, kidney, and heart and at moderate levels in colon, small intestine, and ovary. Classical ACE inhibitors such as captopril and lisinopril do not inhibit ACE-2 activity. Novel peptide inhibitors of ACE-2 do not inhibit ACE activity. Genetic data from Drosophila, mice and rats show that ACE-2 is an essential regulator of heart function in vivo.

ACE2 has been shown to be a functional receptor of the human coronaviruses SARS-CoV and SARS-CoV-2. This human anti-ACE2 antibody (catalog #AF933) was used to block the variant SARS-CoV-2 and ACE2 interaction to elucidate viral transmission and potential therapeutic strategies. In some embodiments, the substrate-bound test antibody and/or the free test antibody may comprise the human anti-ACE2 antibody at the corresponding ACE2 binding site. However, embodiments include analogous rabbit, goat, or horse antibodies. In some embodiments, antibodies from the Immunoglobulin G and/or Immunoglobulin M families may be used.

ACE-2 is the host cell receptor responsible for mediating infection by SARS-CoV-2, the novel coronavirus responsible for coronavirus disease 2019 (COVID-19). Treatment with anti-ACE-2 antibodies disrupts the interaction between virus and receptor.

It has been reported that ACE2 is the main host cell receptor of 2019-nCoV and plays a crucial role in the entry of virus into the cell to cause the final infection. To investigate the potential route of 2019-nCov infection on the mucosa of oral cavity, bulk RNA-seq profiles from two public databases including The Cancer Genome Atlas (TCGA) and Functional Annotation of The Mammalian Genome Cap Analysis of Gene Expression (FANTOM5 CAGE) dataset were collected. RNA-seq profiling data of 13 organ types with para-carcinoma normal tissues from TCGA and 14 organ types with normal tissues from FANTOM5 CAGE were analyzed in order to explore and validate the expression of ACE2 on the mucosa of oral cavity. Further, single-cell transcriptomes from an independent data generated in-house were used to identify and confirm the ACE2-expressing cell composition and proportion in oral cavity. The results demonstrated that the ACE2 expressed on the mucosa of oral cavity. Interestingly, this receptor was highly enriched in epithelial cells of tongue. Preliminarily, those findings have explained the basic mechanism that the oral cavity is a potentially high risk for 2019-nCoV infectious susceptibility and provided a piece of evidence for the future prevention strategy in dental clinical practice as well as daily life.

ACE2 expression was higher in tongue than buccal and gingival tissues. These findings indicate that the mucosa of oral cavity may be a potentially high risk route of 2019-nCov infection.

It may have been found that the mean expression of ACE2 was higher in oral tongue (13 tissues) than others (19 tissues), while may due to the limitation of the sample size, the p value was not significant (P=0.062).

ACE2 may be expressed in oral tissues (0.52% ACE2-positive cells), and higher in oral tongue than buccal and gingival tissues (95.86% ACE2-positive cells located in oral tongue). The ACE2-positive cells could be found in oral tissues including epithelial cells (1.19% ACE2-positive cells), T cells (<0.5%), B cells (<0.5%), and fibroblast (<0.5%), and the ACE2 was highly enriched in epithelial cells, of which 93.38% ACE2-positive cells belong to epithelial cells. The above results indicated that the ACE2 could be expressed on the epithelial cells of the oral mucosa and highly enriched in tongue epithelial cells.

Furthermore, we have also demonstrated that the ACE2-positive cells were enriched in epithelial cells, which was also reported by previous study. These findings indicated that oral cavity could be regarded as potentially high risk for 2019-nCov infectious susceptibility.

Stomach and Intestines have very high ACE2 levels, hence stomachache and diarrhea associated with infection by SARS-CoV-2.

The ACE2-expressing cells in oral tissues, especially in epithelial cells of tongue, might provide possible routes of entry for the 2019-nCov, which indicate oral cavity might be a potential risk route of 2019-nCov infection. Those preliminary findings have explained the basic mechanism that the oral cavity is a potentially high risk for 2019-nCoV infectious susceptibility and provide a piece of evidence for the future prevention strategy in clinical practice as well as daily life.

ACE-2 is a type I transmembrane metallocarboxypeptidase with homology to ACE, an enzyme long-known to be a key player in the Renin-Angiotensin system (RAS) and a target for the treatment of hypertension. It is mainly expressed in vascular endothelial cells, the renal tubular epithelium, and in Leydig cells in the testes. PCR analysis revealed that ACE-2 is also expressed in the lung, kidney, and gastrointestinal tract, tissues shown to harbor SARS-CoV. The major substrate for ACE-2 is Angiotensin II. ACE-2 degrades Angiotensin II to generate Angiotensin 1-7, thereby, negatively regulating RAS. ACE-2 has also been shown to exhibit a protective function in the cardiovascular system and other organs.

ACE-2 is an Entry Receptor for SARS-CoV-2.

Based on the sequence similarities of the RBM between SARS-CoV-2 and SARS-CoV, several independent research groups investigated if SARS-CoV-2 also utilizes ACE-2 as a cellular entry receptor. Zhou et al. showed that SARS-CoV-2 could use ACE-2 from humans, Chinese horseshoe bat, civet cats, and pigs to gain entry into ACE-2-expressing HeLa cells. Hoffmann et al. reported similar findings for human and bat ACE-2. Additionally, Hoffmann et al. showed that treating Vero-E6 cells, a monkey kidney cell line known to permit SARS-CoV replication, with an Anti-ACE-2 Antibody (R&D Systems, Catalog #AF933) blocked entry of VSV pseudotypes expressing the SARS-CoV-2 S protein.

Inhibiting TMPRSS2 Activity May Block SARS-CoV-2 Entry

For SARS-CoV entry into a host cell, its S protein needs to be cleaved by cellular proteases at 2 sites, termed S protein priming, so the viral and cellular membranes can fuse. Specifically, S protein priming by the serine protease TMPRSS2 is crucial for SARS-CoV infection of target cells and spread throughout the host. Hoffmann et al. investigated if SARS-CoV-2 entry is also dependent on S protein priming by TMPRSS2. Treatment of the Calu-3 human lung cell line with the serine protease inhibitor camostat mesylate partially blocked entry of VSV pseudotypes expressing the SARS-CoV-2 S protein. Similar effects of camostat mesylate treatment were seen with primary human lung cells and with Calu-3 cells incubated with authentic SARS-CoV-2. In embodiments of the present invention, the S proteins used may be cleaved before reaction in the reaction chamber of the device. In other embodiments, TMPRSS2 may be introduced as a priming agent into the reaction chamber before the methods herein are implemented.

In a healthy person, the ACE2 receptor chops up two forms of a protein called angiotensin to keep blood pressure stable, among other things. SARS and the novel coronavirus, however, use the receptor to infiltrate cells. The virus can latch onto ACE2 and sneak inside, replicating itself inside the cell and then wreaking havoc throughout the body.

Normally, ACE2 is found on lung, kidney, heart, and gut cells. However, ACE2 may also be found in nasal cells and buccal cells and may be even higher in tongue cells. Higher levels of ACE2 may be a risk factor for people regarding susceptibility to Covid-19. Individuals with smoking-damaged lungs and those with chronic obstructive pulmonary disease (COPD) may be likely to have a higher level of the angiotensin-converting enzyme II (ACE2). However, other risk factors for individuals includes diabetes, heart failure, high blood pressure, and immunological disorders.

ACE-2 is an enzyme similar in function to angiotensin-converting enzyme (ACE), but different in that it not only (like ACE) converts angiotensin I to angiotensin II, which is a potent vasoconstrictor, but also further degrades angiotensin II to angiotensin (1-9) and angiotensin (1-7). These are potent vasodilators and could even be inhibitors of the vasoconstrictor renin-angiotensin system. ACE-2 is present in upper respiratory mucosa, lung mucosa, heart muscle, and gut lining. It seems to be an important molecule in the regulation of cardiovascular function.

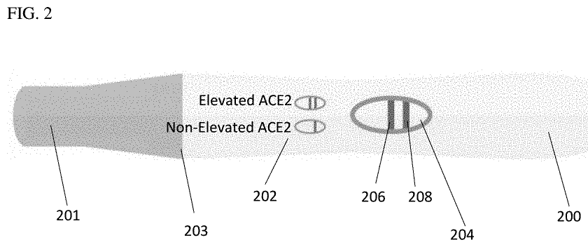

FIG. 2 depicts a diagram of a Covid-19 susceptibility testing device 200. For example, some embodiments of the device may comprise a home test device for detection of elevated levels of ACE2 102 in accordance with the principles of the present invention. The device 200 may comprise a receiving chamber 201 configured to receive a human bodily fluid, such as saliva, urine, blood serum or any other bodily fluid in which an analyte thereof may be coordinated to Covid-19 susceptibility. Further, the device may comprise a reaction medium configured to indicate Covid-19 susceptibility when concentration of an analyte exceeds a predetermined threshold in the human bodily fluid. The reaction medium may be a solution and/or a fixed substrate. By way of example, the device may include both a control chamber and a test chamber. Alternatively, the device may comprise a fixed substrate with bound proteins corresponding to a control portion and a test portion. In embodiments comprising a filtering step, the filtering may be performed in a separate chamber, with a separate filtering strip in the receiving chamber, or in the reaction and/or control chambers with washing away undesirable particles and/or sequestering undesirable particles to a portion of the reaction medium in which the undesirable particles do not interfere with the Covid-19 susceptibility indication. In some embodiments, the receiving chamber may receive the fluid and a filter may be positioned between the receiving chamber and the test and/or control chambers. In some embodiments, the filter may be embodied on a chromatography-style substrate in which the human bodily fluid must pass through the substrate, including the filter to reach the reaction and control chambers or sections. In some embodiments, the filter section may be coated with a binding antibody to prevent the lateral flow of ACE2 such that the average amount of ACE2 is bound and does not reach the reaction chamber. In these embodiments, only an excess amount of ACE2 reaches the reaction chamber for chromogenic indication of Covid-19 susceptibility.

Thus, a method of testing for Covid 19 susceptibility can include providing a first protein at a control indicating site, wherein the first protein may comprise a binding affinity for the chromogenic control protein and providing a second protein at a test indicating site, wherein the second protein may comprise a binding affinity for bodily proteins that correspond to elevated ACE2. For example, the second binding protein may comprise a binding affinity for ACE2 as integrated in a cell. In these embodiments, an indicator fluid that may comprise a third protein, such as a chromogenic indicator, configured to bind cellular ACE2 may be bound at the test indicating site to indicate Covid-19 susceptibility. In some embodiments, the second protein may comprise a binding affinity for at least a portion of ACE2 protein.

The device may indicate Covid-19 susceptibility by color change. For example, a chromogenic compound may be included in the reaction chamber or the reaction substrate to provide the color change indication of Covid-19 susceptibility. In some embodiments, the chromogenic compound may be at least one of ninhydrin, a ninhydrin reaction product, dinitrophenol, a dinitrophenol reaction product, paranitroaniline, and a paranitroaniline reaction product, or any congener of these listed chromogenic compounds. However, embodiments may include any chromogenic indicator that may react with ACE2, Leucin, or any portion of ACE2.

In some embodiments, the reaction may indicate Covid-19 susceptibility at a predetermined reaction time. Example times include seven minutes, ten minutes, and 20 minutes.

In some embodiments, the human bodily fluid may be tested for presence of an analyte or presence of the analyte above a predetermined threshold to determine Covid-19 susceptibility. The analyte may include ACE2 in a cell, free ACE2, reaction products of ACE2, etc. For example, Angiotensin I may be combined with ACE2 to break down the ACE2 into Leucine. The presence of Leucine or the concentration of Leucine above the predetermined threshold may be used to indicate susceptibility to Covid-19. For example, the analyte, especially Leucine, may be present in the human bodily fluid before digestion of ACE2. In these embodiments, an additional step of filtering the analyte from the human bodily fluid before digesting the ACE2 may be performed. The filter may selectively bind to one or more of pathogens, free proteins, amino acids, and small molecules. In embodiments in which Leucine is filtered before introduction of the sample to the reaction medium, the remaining filtered human bodily fluid may then be reacted with Angiotensin I such that the Leucine present after digestion of ACE2 corresponds to the amount of ACE2 in the sample. Thus, ACE2 or Leucine may correspond by absolute presence and/or by exceeding a predetermined threshold concentration to the determination of Covid-19 susceptibility due to excess ACE2 in the human bodily fluid sample.

The Covid-19 susceptibility testing device may comprise a reaction medium of a substrate-bound test antibody comprising an ACE2 binding affinity. In these embodiments, a small molecule capable of binding ACE2's binding site may be used. For example, a Histidine-Leucine-like dipeptide, such as Histidine-Leucine dipeptide or a Histidine-Leuicine congener may be used. In some embodiments, a carboxylate group capable of serving as a zinc coordinating element, such as a protein containing Ala-Pro-Lys-paranitroaniline, a protein containing Ala-Pro-Lys-dinitrophenol, or another similar protein or small molecule may be used for binding ACE2, free or cellular ACE2, and then subsequent reaction to provide color change indicating present of ACE2, presence of ACE2 above the predetermined threshold, and thus Covid-19 susceptibility.

In some embodiments, the test analyte of the human bodily fluid may be ACE2, the predetermined threshold of the analyte may be 75.24 ng/ml, and the human bodily fluid may be urine. In further embodiments, the test analyte may be ACE2, the predetermined threshold of the analyte may be 685.1 ng/ml, and the human bodily fluid may be serum. Further embodiments include that the test analyte may be Leucine, the predetermined threshold of the analyte may be 115 pg/ml, and the human bodily fluid may be urine. Embodiments also include the test analyte may be Leucine, the predetermined threshold of the analyte may be 105 ng/ml, and the human bodily fluid may be serum. Additional embodiments include the test analyte may be 2,4-dinitrophenol, the predetermined threshold of the analyte may be 161 pg/ml, and the human bodily fluid may be urine.

In embodiments comprising a substrate-based reaction medium, the device may comprise a substrate-bound test antibody on the test line and/or the control line. For example, the substrate-bound test antibody may comprise an affinity for ACE2 or its byproducts, such as Leucine. The substrate-bound control antibody on the control line may comprise an affinity for the substrate-bound control antibody comprising an affinity for a free control antigen comprising a colorimetric enzyme. The device may include a blister comprising a fluid comprising free test antibody comprising the colorimetric enzyme and the free test antibody having an affinity for ACE2 and the fluid further comprising the free control antigen. Thus, the blister may withhold the fluid from the reaction chamber until compromised upon the desired testing time, typically after the human bodily fluid is received in the chamber of the device. The control line may change color upon introduction of the fluid to the reaction chamber and the test line may change color upon introduction of the fluid to the reaction chamber when elevated ACE2 is present in the received bodily fluid in the reaction chamber.

The device 200 may further comprise a key 202 for comparison of test results. The device 200 may comprise a reaction chamber 204 for housing the reaction and/or obtaining test results. For example, test results may comprise a control line (or control site) 208. However, lack of a control line or positive indication at the control site 208 may indicate an invalid test result. On the other hand, inclusion of a variable line at the test chamber (or test site) 206 may indicate elevated levels of ACE2 102 in the reaction chamber. In some embodiments, the reaction chamber 204 may receive a bodily fluid, such as saliva, a tongue scraping, serum, blood, and/or urine. A blister (not depicted) may also comprise one or more reaction fluids to be introduced into the reaction chamber. In some embodiments, the human bodily fluid is received in the receiving chamber 201 and may travel to the reaction chamber 204, such as through a chromatography medium. In such embodiments, a filter 203 may be positioned between the receiving chamber 201 and the reaction chamber 204. The filter 203 may comprise a binding affinity for pathogens, proteins, or other debris such that interference is reduced at the reaction chamber. In further embodiments, the filter 203 may have an affinity for an analyte such that the average amount of analyte is filtered out and only excess analyte above the population average is shown as a positive at test site 206.

The color change may be effected by antibody-specific binding affinity causing chromogenic change when bound. Example colors include blue (e.g. via involvement of beta-galactosidase and Xgal), purple (e.g. purple acid phosphatase oxidation by hydrolysis of phosphate esters and anhydrides under acidic conditions), green (e.g. bromocresol green when positive result causes pH about 4.8 at the substrate), red (phenol red when a positive result causes pH 6.8 to 8.2), pink (phenol red when positive result causes pH greater than 8.2 at the substrate), red (methyl red when positive result causes pH under 4.4 at the substrate, but yellow in pH greater than 6.2, and orange between pH 4.4 and 6.2), black (Eriochrome Black T, produced by Huntsman Petrochemical, LLC, when deprotonated at the substrate during a positive result or red when complexed with calcium or magnesium during positive result at the substrate), or other known colorimetric enzymes, indicators, and byproducts. For simplicity, blue and beta-galactosidase will be described in the examples following. In examples using beta-galactosidase as the chromogenic enzyme, X-gal (also abbreviated BCIG for 5-bromo-4-chloro-3-indolyl-β-D-galactopyranoside) may also be introduced into the reaction chamber 204, such as by the blister. X-gal, when cleaved by β-galactosidase, yields galactose and 5-bromo-4-chloro-3-hydroxyindole. The latter then spontaneously dimerizes and is oxidized into 5,5′-dibromo-4,4′-dichloro-indigo, an intensely blue product which is insoluble. X-gal itself is colorless, so the presence of blue-colored product may therefore be used as a test for the presence of active β-galactosidase. However, if X-gal is stored with the unbound test enzyme and control enyzyme, the β-galactosidase must be in an inactive tertiary conformation until the binding of the bound control antibody and/or binding of the ACE2 102 on the cell 100.

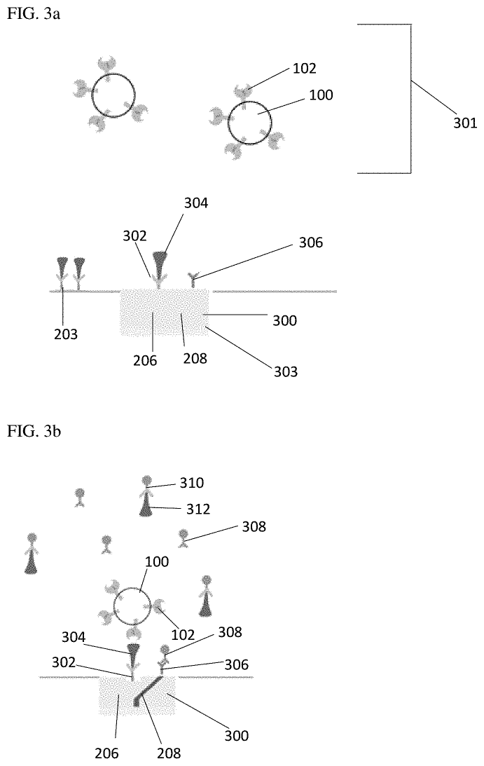

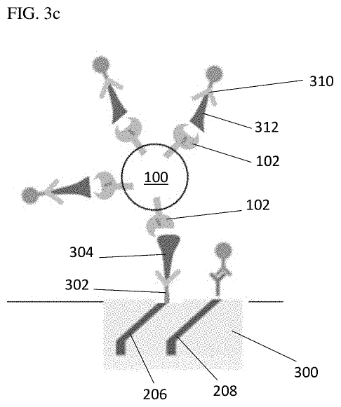

FIG. 3a depicts a diagram of ACE2-containing human cells 100 in suspension above a substrate-bound test antibody 302 comprising an S-protein 304 in accordance with the principles of the present invention. This suspension may occur in the reaction chamber 204 after the human bodily fluid 301 is received in the reaction chamber 204. By diffusion, the ACE2 102 may bind the human cell 100 to the substrate-bound S-protein 304. The attached human cell 100 may then become attached to a substrate 300, such as through binding of the substrate-bound S-protein 304 (e.g. substrate-bound test antibody). Example substrates include polyethylene. In some embodiments, the substrate-bound test antibody 302 may be chromogenic and/or may bind a chromogenic enzyme. A substrate-bound control antibody 306 may also be bound to the substrate 300. The substrate-bound control antibody 306 may not necessarily yet be bound in such a manner as to generate color change of the control site 208. In some embodiments, the substrate-bound control antibody 306 may be chromogenic when bound and/or the substrate-bound control antibody 306 may have a binding affinity for a chromogenic enzyme. At this stage, both the control site 208 and the test site 206 may be unchanged.

In some embodiments, antibodies 302, 306, 308, and/or 310 may comprise and/or be bound to additional proteins and/or enzymes for creating binding affinities and/or carrying out the mechanisms described herein.

Further embodiments include filter 203 positioned before the human bodily fluid 301 reaches the test site 206 and the control site 208. For example, filter 203 may selectively bind extraneous proteins, amino acids, and/or pathogens. The filter 203 may be configured to selectively bind and remove the amount of analyte (e.g. ACE2, Leucine, etc.) found in a predetermined amount (e.g. 10 drops, 10 ml, etc.) of human bodily fluid. Thus, by removing the average amount of analyte in the population, only excess analyte may reach test site 206 to provide a positive result. By way of example, the reaction medium 303 may be a solid chromatography-type strip over which the human bodily fluid 301 passes. However, embodiments include the reaction medium 303 being fluid or liquid additives in the respective one or more chambers (e.g. the receiving chamber may be the reaction chamber with the optional step of filtering and introduction of further reactants).

FIG. 3b depicts a diagram of the ACE2-containing human cells 100 bound to the S-protein 304 of the substrate-bound test antibody 302 with the addition of enzyme-containing free test antibody 310 bound to S-proteins 312 in accordance with the principles of the present invention. A free control antibody 308 bound to a chromogenic enzyme may also be introduced in which the free control antibody 308 may have a binding affinity to the substrate-bound control antibody 306. This binding affinity may occur by antibody affinity in which free control antibody 308 and substrate-bound control antibody 310 may be complementary antibodies and/or through antibody-antigen interactions. Introduction of the free test antibody 310 and/or the free control antibody 308 may occur by popping a blister pack situated beside the reaction chamber 204 such that these elements are introduced to the reaction chamber 204. After this occurs, the free control antibody 308, which may have a specific affinity for the substrate-bound control antibody 306, may bind the substrate-bound control antibody 306. After such binding, the bound control antibody, free control antibody, and/or a bound enzyme thereof may exhibit a color change at the control site 208, as indicated on control site 208. At this stage, many of the cells 100 may be collected and bound on the substrate at the site of test line 206. Free ACE2 102 on the cells 100 may bind to the free test antibody 310, such that the free test antibody 310 may become concentrated at test line 206. However, before the free test antibody 310 is bound, test line 206 may not necessarily indicate color change. In this manner, the integrity of the control enzyme and indicator (e.g. Xgal) may be tested.

In some embodiments, the substrate-bound test antibody 302 and/or the free test antibody 310 may comprise the human anti-ACE2 antibody (R&D Systems, Catalog #AF933) at the corresponding ACE2 binding site rather than S-proteins 304 and 312. Therefore, the S-proteins are provided by way of example for illustration herein and may be referred to as ACE2 affinity sites and/or ACE2 binding sites 304 and 312, which may comprise S-proteins, anti-ACE2 antibodies, and/or any other protein and/or or RNA structure having binding affinity for human ACE2 receptors. However, embodiments include analogous rabbit, goat, or horse antibodies used at these affinity sites for binding to ACE2. In some embodiments, antibodies from the Immunoglobulin G and/or Immunoglobulin M families may be used. The free test antibody 310 may further comprise a chromogenic enzyme. In some embodiments, this chromogenic enzyme may become activated upon binding of the S-protein 312. The free control antibody 308 may comprise a chromogenic enzyme. In some embodiments, this chromogenic enzyme may become activated upon binding to the substrate-bound control enzyme 306.

FIG. 3c depicts a diagram of ACE2 containing human cells 100 bound to the substrate 300 and further bound to the S-proteins 312 of enzyme-containing free test antibodies 310 in accordance with the principles of the present invention. Binding of the free test S-protein 312 of the free test antibody 310 may further activate a chromogenic enzyme on the free test antibody 310. The concentration of bound test antibodies at test line 206 may provide a color change at test line 206. However, embodiments include that the substrate-bound test antibody 302 may be chromogenic such that color change is effected when ACE2 is bound to it's substrate-bound S-protein 304. In such embodiments, the ACE2 and/or ACE2 containing cells may be detected as present and/or elevated. In some embodiments, the Xgal product may deposit onto the PE at test site 206 and/or control site 208 due to insolubility.

In some embodiments, the human cell 100 may not necessarily be bound as an antigen. In these embodiments, the substrate-bound test antibody may have an affinity for a first binding site of free ACE2 and the free test antibody may have an affinity for a second biding site of free ACE2, such that the substrate-bound test antibody sandwiches the free ACE2 with the free test antibody. This may then result in color change through a chromogenic enzyme, such as by localizing at the test line 206 and/or activating the chromogenic enzyme of the free test antibody after binding.

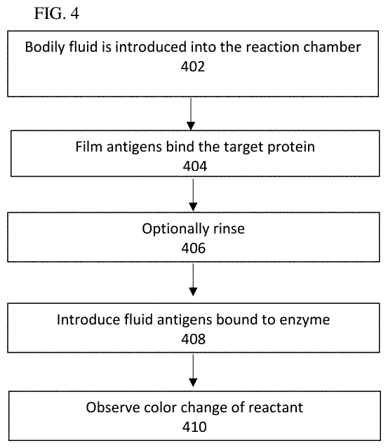

FIG. 4 depicts a block-level diagram of a method of testing for elevated ACEII in an individual as illustrated in FIGS. 3a-3c in accordance with the principles of the present invention. In step 402, human bodily fluid, such as serum, saliva, blood, urine, and/or a tongue scraping may be introduced into the reaction chamber 204. Optionally, a filter, such as filter 203, may be introduced to the human bodily fluid to remove undesirable components thereof. Step 404 may include film test antibodies, such as substrate-bound test antibody 302, binding the target ACE2 proteins, such as through the substrate-bound S-protein 304.

In step 406, after sufficient binding time has elapsed, the reaction chamber may be rinsed with a fluid that does not contain ACE2 containing cells to reduce competitive binding and increase result accuracy. For example, elapsed binding time may be 30 seconds, 1 minute, or 5 minutes.

In step 408, fluid antibodies may be introduced. This step may be performed by popping a blister that introduces the fluid antibodies, such as the free control antibody 308 and free test antibody 310, into the reaction chamber 204. In some embodiments, the free antibodies 308 and 310 may be bound to an enzyme that may not necessarily react with the chromic reactant, such as Xgal, until binding and tertiary conformation change. After binding, the Xgal, or other chromic reactant, may be consumed by the chromic enzyme until color is produce at one or both test sites (206 and/or 208). However, embodiments include active enzyme, even when unbound, such that the chromic reactant (e.g. Xgal) may be stored in a second chromic reactant blister. This blister may be popped to add the chromic reactant after binding is complete in the reaction chamber 204.

In step 410, the color change of the reactants may be observed, such as for concentration at control site 206 and/or test site 208. As color change may occur based on presence of ACE2 receptors, the intensity and speed of reaction may be key to distinguishing presence of elevated levels of ACE2 receptors in some embodiments. Therefore, observing that the test site 208 remains at least as intense, thick, and/or dark as the control line 206 at any given time may be key to the determination of elevated ACE2 receptors. In some embodiments, the control line 206 may be representative of average, a first standard deviation, and/or a second standard deviation of ACE2 receptor concentration in the population. Observing the reaction speed may also be key, as the endpoint may be similar with or without elevated ACE2. Therefore, the reaction may be compared at 10 seconds, 30 seconds, 1 minute, and/or 5 minutes. In some embodiments, lack of color change and/or weak color change, especially relative to the standard, may be indicative of lack of elevated ACE2 receptors.

By way of example, 2.18, 2.44, and/or 3.00 ng/ml ACE2 or higher may be considered elevated levels of ACE2 in urinary bodily fluid. The color change of control site 208 may be calibrated to correlate to one or more of these ACE2 concentrations such that test line 206 may indicate faster reaction, deeper color change, etc. when higher than these levels.

If elevated ACE2 receptors are determined, the individual's physician may be contacted. The individual may be prescribed a prophylaxis to help prevent Covid-19, such as hydroxychloroquine, azithromycin, and/or zinc. However, the age of the individual may be determinative of whether to prescribe prophylaxis. For example, 24% of serious infection outcomes may be due to age. Further risk factors may be considered, such as diabetes, heart conditions, high blood pressure, COPD, asthma, and/or immunological conditions. In some embodiments, prophylaxis may be prescribed and/or elevated ACE2 reported when the individual is 80 years old or greater and/or has three underlying health conditions.

Further embodiments include the introduction of an enzyme, such as in step 408, to digest ACE2 into one or more amino acids, such as Leucine. By way of example, angiotensin I may cleave ACE2 to produce Leucine in the solution. In the test chamber and/or at the test site, the presence of Leucine and/or the presence of Leucine above a predetermined threshold may be indicated colorimetrically for a positive result of Covid-19 susceptibility. These may be accomplished by the substrates and enzymes mentioned herein.

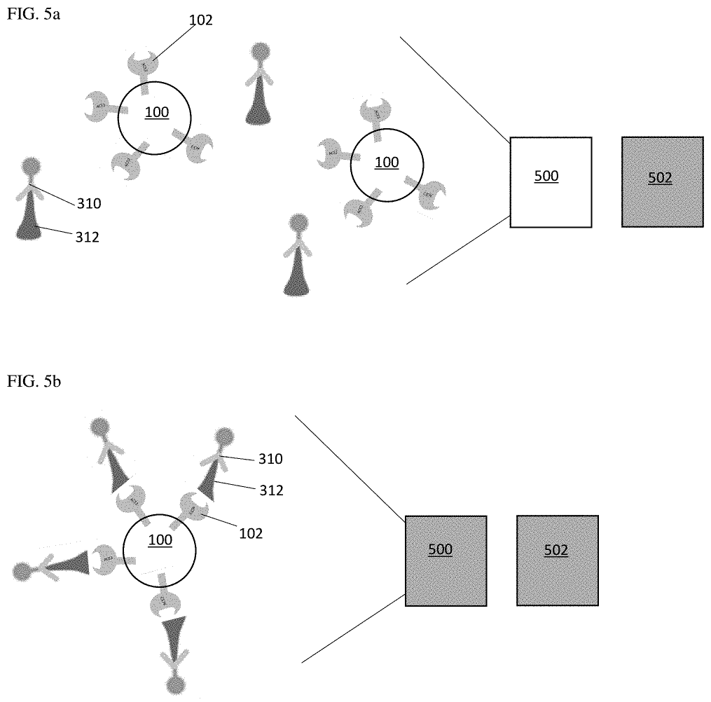

FIG. 5a depicts a diagram of an alternate embodiment of a reaction mechanism for testing for elevated ACE2 of an individual in accordance with the principles of the present invention. In these embodiments, bodily fluid, such as saliva and/or a tongue scraping may be collected into reaction chamber 500. Control chamber 502 may contain a set unchangeable color as a standard. Embodiments include free control antibody 308 and an uncolored substrate the free control antibody 308 may comprise a chromogenic enzyme that may cause a color change after introduction of a reactant, such as Xgal, e.g. by popping a blister pack into control chamber 502. After bodily fluid is introduced into test chamber 500, the cells 100 may bind free test antibody 310.

FIG. 5b depicts a diagram of the alternate embodiment reaction mechanism of FIG. 5a having a positive result in accordance with the principles of the present invention. In some embodiments, a blister pack containing chromogenic reactant, may be popped into reaction chamber 500 and/or control chamber 502. In embodiments that introduce chromogenic reactant to both chambers, popping the blister may introduce chromogenic reactant to both chambers at the same time. The enzyme in the control chamber 502 may be set to indicate and/or correlated with a control level, such as representative of average population, first standard deviation elevated, and/or second standard deviation elevated ACE2. In the test reaction chamber 500, the cell 100 ACE2 102 may bind free test S-protein 312 of the free test antibody 310. The binding of the free test antibody 310 may cause tertiary confirmation change such that a bound chromogenic enzyme is activated. The chromogenic enzyme may consume a chromogenic reactant, such as Xgal, such that a color is generated that corresponds to the level of ACE2 in the test reaction chamber 500. The chromogenic reactant may be added to the test reaction chamber 500 and/or the control chamber 502 in step 604. The color change intensity, duration, and/or speed may be observed against the control chamber 502 after an elapsed reaction time in step 608, such as at 10 seconds, 30 seconds, 1 minute and/or 5 minutes, for the determination of elevated ACE2. In step 610, the standard may be used for determination of ACE2 elevation based on color intensity, duration, and/or reaction speed.

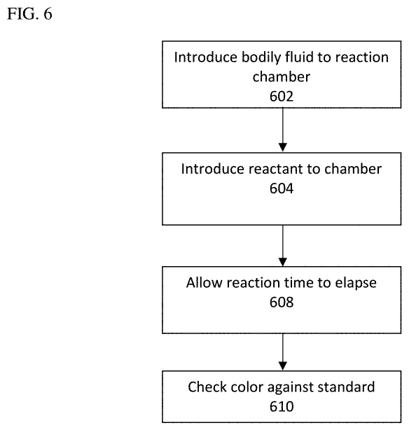

FIG. 6 depicts a block-level diagram of the method of the alternate embodiment of testing depicted in FIGS. 5a and 5b in accordance with the principles of the present invention. In these embodiments, the reaction medium 303 may be fluid in the one or more respective chambers. In step 602, bodily fluid may be introduced into the test reaction chamber 500. Optionally, a filter, such as filter 203, may be introduced to the human bodily fluid to remove undesirable components thereof. If cells 100 are present with ACE2 102, then binding of the free test antibody 310 may occur. In turn, the bound enzyme of the free test antibody 310 may become activated. Chromogenic reactant may be present and/or added for observable color change in step 604. In some embodiments, lack of color change and/or weak color change, especially relative to the standard, may be indicative of lack of elevated ACE2 receptors.

If elevated ACE2 receptors are determined, the individual's physician may be contacted. The individual may be prescribed a prophylaxis to help prevent Covid-19, such as hydroxychloroquine, azithromycin, and/or zinc. However, the age of the individual may be determinative of whether to prescribe prophylaxis. For example, 24% of serious infection outcomes may be due to age. Further risk factors may be considered, such as diabetes, heart conditions, high blood pressure, COPD, asthma, and/or immunological conditions. In some embodiments, prophylaxis may be prescribed and/or elevated ACE2 reported when the individual is 80 years old or greater and/or has three underlying health conditions.

In some embodiments, the products, such as angiotensis (1-7) of ACE2 may be bound by the test antibodies and color change induced by increased levels that correlated to increased levels of ACE2. For example, higher levels of angiotensis (1-7) may indicate increase ACE2 of the individual. Color change may be calibrated such that the indicators show color change at higher than average, first standard deviation, or second standard deviation of ACE2 receptors based on binding of angiotensis (1-7) for activation of the color change enzyme at the test line binding site.

Further embodiments include the introduction of an enzyme, such as in step 604, to digest ACE2 into one or more amino acids, such as Leucine. In the test chamber, the presence of Leucine and/or the presence of Leucine above a predetermined threshold may be indicated colorimetrically for a positive result of Covid-19 susceptibility. These may be accomplished by the substrates and enzymes mentioned above.

What is claimed is:

1. A Covid-19 susceptibility testing device comprising:
a receiving chamber configured to receive a human bodily fluid; and
a reaction medium configured to indicate Covid-19 susceptibility when concentration of an analyte exceeds a predetermined threshold in the human bodily fluid.

2. The Covid-19 susceptibility testing device of claim 1, wherein the human bodily fluid comprises one of saliva, urine, and serum.

3. The Covid-19 susceptibility testing device of claim 1, wherein the reaction medium indicates Covid-19 susceptibility by color change.

4. The Covid-19 susceptibility testing device of claim 3, wherein a chromogenic compound provides the color change indication of Covid-19 susceptibility, the chromogenic compound comprising at least one of ninhydrin, a ninhydrin reaction product, a ninhydrin congener, dinitrophenol, a dinitrophenol reaction product, a dinitrophenol congener, paranitroaniline, a paranitroaniline reaction product, and a paranitroaniline congener.

5. The Covid-19 susceptibility testing device of claim 1, further comprising Angiotensin I to produce Leucine from ACE2 in the human bodily fluid, wherein Leucine is the analyte.

6. The Covid-19 susceptibility testing device of claim 1, wherein the reaction medium comprises a substrate-bound test antibody comprising an ACE2 binding affinity.

7. The Covid-19 susceptibility testing device of claim 1, wherein the test analyte is ACE2, the predetermined threshold of the analyte is about 75.24 ng/ml, and the human bodily fluid is urine.

8. The Covid-19 susceptibility testing device of claim 1, wherein the test analyte is ACE2, the predetermined threshold of the analyte is about 685.1 ng/ml, and the human bodily fluid is serum.

9. The Covid-19 susceptibility testing device of claim 1, wherein the test analyte is Leucine, the predetermined threshold of the analyte is about 115 pg/ml, and the human bodily fluid is urine.

10. The Covid-19 susceptibility testing device of claim 1, wherein the test analyte is Leucine, the predetermined threshold of the analyte is about 105 ng/ml, and the human bodily fluid is serum.

11. The Covid-19 susceptibility testing device of claim 1, wherein the test analyte is 2,4-dinitrophenol, the predetermined threshold of the analyte is 161 about pg/ml, and the human bodily fluid is urine.

12. A method of testing for Covid-19 susceptibility in a subject, comprising:
receiving a human bodily fluid; and
combining the bodily fluid with a reaction medium configured to indicate Covid-19 susceptibility in the subject when an analyte in the human bodily fluid exceeds a predetermined threshold.

13. The method of testing for Covid-19 susceptibility of claim 12, wherein the human bodily fluid comprises one of saliva, urine, and serum.

14. The method of testing for Covid-19 susceptibility of claim 12, wherein the reaction medium indicates Covid-19 susceptibility by color change.

15. The method of testing for Covid-19 susceptibility of claim 12, wherein the test analyte is ACE2, the predetermined threshold of the analyte is about 75.24 ng/ml, and the human bodily fluid is urine.

16. The method of testing for Covid-19 susceptibility of claim 12, wherein the test analyte is ACE2, the predetermined threshold of the analyte is about 685.1 ng/ml, and the human bodily fluid is serum.

17. The method of testing for Covid-19 susceptibility of claim 12, wherein the test analyte is Leucine, the predetermined threshold of the analyte is about 115 pg/ml, and the human bodily fluid is urine.

18. The method of testing for Covid-19 susceptibility of claim 12, wherein the test analyte is Leucine, the predetermined threshold of the analyte is about 105 ng/ml, and the human bodily fluid is serum.

19. The method of testing for Covid-19 susceptibility of claim 12, wherein the test analyte is 2,4-dinitrophenol, the predetermined threshold of the analyte is about 161 pg/ml, and the human bodily fluid is urine.

20. A kit comprising:
a receiving chamber capable of receiving bodily fluid; and
a reaction medium configured to indicate Covid-19 susceptibility when concentration of an analyte exceeds a predetermined threshold in the human bodily fluid.
